(19) United States
(12) Patent Application Publication
(10) Pub. No.: US 2008/0153173 A1
(43) Pub. Date: Jun. 26, 2008

(54) Method and Device for Determining the Concentration of Organically Bound Halogens

(76) Inventor: Christian Buck, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/817,047

(22) PCT Filed: Feb. 24, 2006
(86) PCT No.: PCT/EP2006/001717

§ 371 Date: Aug. 24, 2007

(30) Foreign Application Priority Data
Feb. 25, 2005 (DE) 20 2005 003 171.2
Apr. 23, 2005 (DE) 20 2005 006 570.6
Jul. 4, 2005 (DE) 20 2005 010 595.3
Jul. 12, 2005 (DE) 20 2005 010 988.6

(51) Int. Cl. G01N 33/00; G01N 21/64; G01N 21/01

(52) U.S. Cl. 436/125; 422/82.05; 422/82.08

(57) ABSTRACT

In a method for determining the concentration of organically bound halogens present in waste water, a pre-specified amount of waste water is withdrawn and converted into a measurement sample. A spectral measurement is subsequently carried out on the measurement sample, and the result of the spectral measurement is compared with reference measurements in order to determine the concentration of organically bound halogens. The organically bound halogens are enriched in the measurement sample by extraction or adsorption. Inorganically bound halogens are removed from the measurement sample. The comparison of the spectral measurement with reference measurements is carried out using chemometric methods. An automatic measuring device 9 has a device 10 for taking an amount of sample from a flow system, a device 11 for conversion of the amount of sample into a measurement sample, a device 12 for carrying out spectral measurements on the measurement sample, and an evaluation device 13 for evaluation of the result of the spectral measurements using reference measurements. The measuring device also has a device for the automatic provision of an amount of sample and a device 11 for the automatic transfer of the measurement sample into the device 12 for carrying out the spectral measurement. The measuring device can also be used for carrying out a determination of the concentration of other spectroscopically active substances, in particular the concentrations of AOX contributors, BTEX contributors, alcohols, peroxides, metals, aldehydes, ketones, polymers, aromatics, functionalised aromatics or polyaromatics.

The invention relates to a method for determining the concentration of organically bound halogens or the like present in flow systems, such as, for example, waste water.

For clarification and simplification, the invention is described below with reference to the example of the determination of the concentration of organically bound halogens. However, the concentration determination described can also be applied in the same manner directly to the determination of the concentration of other substances in waste water, such as, for example, the concentrations of AOX, EOX, BTEX, alcohols, peroxides, metals, aldehydes, ketones, polymers, aromatics, functionalised aromatics or polyaromatics.

AOX is a sum parameter for adsorbable organically bound halogens whose magnitude is intended to provide information on the toxicity of waste waters. Typical AOX concentrations in waste waters are zero to a few ppm, official limit values are typically set at around one ppm. Correspondingly, EOX is a sum parameter for elutable organically bound halogens, the magnitude of which likewise provides information on waste waters. BTEX is the parameter for the sum of benzene, toluene, ethylbenzene and xylene.

A suitable application example for the use of a method for determining the AOX concentration (or other pollutant concentrations) is an industrial water treatment plant.

The essential constituents of a conventional water treatment plant, such as primary sedimentation tanks, balancing tanks, bioreactors, secondary sedimentation tanks, etc., represent a very large volume. During operation, a plant with 100,000 population equivalents is filled with a few 10,000 m3 of water, typical feed and discharge quantities are a few 100 m3 per hour.

A plant of this type is normally operated continuously and has the job of eliminating nutrients and pollutants from the feed water.

The functioning of the plant and compliance with pre-specified pollutant limit values is monitored by the plant operators themselves and by the authorities by means of measurements. Legally prescribed limit values exist for the maximum discharge concentrations of pollutants; if these are exceeded, considerable additional costs arise and complex purification of the waste water present in the plant becomes necessary. The uncontrolled feed of an excessive concentration of one or more pollutants may also impair or completely destroy the biological equilibrium in the treatment plant, which could cause a reduction in the purification efficiency of the treatment plant and may make temporary shutdown of the treatment plant necessary.

However, potential producers of pollutants, for example in the form of industrial production plants, are often located upstream of such treatment plants and thus, through intentional or unintentional release of pollutants, endanger the functioning and operation of the treatment plant and compliance with officially stipulated discharge limit values with respect to these pollutants.

Particularly unfavourable here is the usually unintentional introduction of pollutants for the reduction or elimination of which the plant is not designed, in particular if this pollution takes place intermittently.

There is therefore a demand for the continuous monitoring of such flow systems with respect to the introduction of pollutants in order to protect the plant itself and the receiving waters (discharge).

Today, AOX determination is usually carried out virtually exclusively using the following method:

Part of the stream to be investigated is branched off continuously from the treatment plant feed over a long period and combined to form a long-term mixed sample (for example 24 h mixed sample). After expiry of the sampling time, the long-term mixed sample is homogenised, and an aliquot thereof is analysed in the laboratory for traces of pollutants using a verifiable method (DIN EN 1485 (H 14) or DIN 38409 (H 22)). The time needed to carry out an AOX measurement in accordance with DIN adds up to about 1.5 hours per sample to be investigated, excluding sample logistics.

In AOX measurement methods of this type, a stipulated amount of active carbon is usually added to an aliquot of a water sample, and the mixture is stirred for one hour. After the stirring, the sample is filtered, the filter cake is freed from inorganic halides by washing with dilute HNO3 and gently dried. The purified filter cake is oxidised completely in a stream of oxygen in a combustion furnace. The adsorbed organically bound halogens are completely mineralised in the process and transferred into a water-filled receiver together with all other oxidation products. The now inorganic halides dissolve therein, and their concentrations can be determined electrochemically. This method is lengthy and thus expensive.

Although this procedure enables the generation of accurate measurement values, it is not capable of the recognition of intermittent pollution of the treatment plant feed in an early and thus timely manner before contamination of the entire treatment plant. Methods for automatic sampling, sample logistics, signal evaluation and signal transmission are capable of drastically shortening the time delay between uptake and a suitable reaction thereto, but the absence of a faster measurement technique for AOX than the DIN methods greatly restricts the effect of such methods.

If it were possible to recognise intermittent introduction of pollutants in good time, it would usually be possible to divert the polluted feed water into a collecting tank provided for this purpose in order to prevent the distribution of the pollutant into the sedimentation tanks of the treatment plant. Other early or immediately initiated measures could also reduce or completely compensate for the introduction of pollutants even during the feed or before entry into sensitive areas of the water treatment plant, enabling contamination of the entire treatment plant to be prevented.

It would therefore be desirable, for example, for the plant operators to be able to receive an immediate preliminary warning, at least for particularly relevant pollutants.

The object of the invention is accordingly to design a measurement method in such a way that rapid and automatable determination of the concentration of AOX and the like is possible.

This invention is achieved by a method for determining the concentration of organically bound halogens present in waste water, where a pre-specified amount of waste water is taken and converted into a measurement sample, a spectral measurement is carried out on the measurement sample, and the result of the spectral measurement is compared with reference measurements in order to determine the concentration of organically bound halogens. Instead of very high precision and lengthy measurement of the concentration of organically bound halogens by means of complex separation of the organically bound halogens and measurement of their concentration by means of electrochemical methods, spectral measurements give rise to an at least approximately correct characteristic quantity for the concentration to be determined. The reference measurements to be carried out in advance are of major importance here since the various AOX contributors may encompass a plurality of substance classes, and the characteristic spectral properties of the individual substances may be very different. Thus, for example, the position, shape and intensity of characteristic emission or absorption lines change as a function of additional moieties or ambient conditions. As soon as information is available on an adequate number of informative and comparable reference measurements, a new spectral measurement can be carried out very quickly with an unknown concentration of AOX contributors and evaluated precisely, and the unknown concentration can be determined.

The spectral measurements and the evaluation thereof can be carried out quickly and in an automated manner. It has been found that the time needed for a measurement of this type, including the requisite withdrawal and preparation of the sample, is about 15 minutes or less, meaning that quasi-continuous recording of the particularly relevant pollutant concentrations is possible.

Individual substances, such as, for example, chlorobenzene, have advantageous optical properties and in particular high optical activity, meaning that the natural concentration of chlorobenzene in flow systems can be determined using commercially available optical spectrometers without complex preparation of the sample or enrichment of chlorobenzene being necessary. It has been found that in such cases, a measurement duration in the region of seconds is sufficient in order to enable informative concentration determinations.

In order to increase the reliability and accuracy of the measurement method, it is advantageous for the organically bound halogens to be enriched in the measurement sample before the spectral measurement is carried out.

An enrichment of this type can be carried out by enriching the organically bound halogens by extraction with a suitable extractant.

Another, likewise advantageous way of improving the detection sensitivity consists in influencing the optical properties of the organically bound halogens by suitable derivatisation thereof. Thus, rapid and simple derivatisation of the substances which contribute to the concentration to be measured could increase their optical activity and thus improve the detection sensitivity of the spectrometer for these substances.

The reliability and accuracy of the measurement method can furthermore be increased by removing inorganically bound halogens from the measurement sample.

According to an advantageous embodiment of the inventive idea, it is provided that the comparison of the spectral measurement with reference measurements is carried out using chemometric methods in order to determine the concentration of organically bound halides present in the waste water.

Chemometry is a mathematical-statistical method for the extraction of physical or chemical information from one- and multidimensional data sets.

Thus, for example, the reproduction of the spectrum of a pure substance A from a set of mixture spectra comprising A is possible if a correlation has been determined in a chemometric method from calibration spectra (spectra of mixtures comprising A in known concentration). Thus, prediction models for the concentration of A in mixtures comprising A can be generated.

Chemometric methods are particularly suitable for the creation of prediction models of AOX contributors, or similar sum parameters, since the optical properties of the many different substances which contribute to the concentration to be determined can each also change as a function of ambient conditions, such as, for example, the temperature, pH, salt content or very generally matrix effects. The work necessary for the creation and evaluation of reference methods can be considerably reduced by the use of chemometric measurements without this causing a corresponding impairment in the concentration determination.

This method can likewise be applied to the determination of the concentration of substance groups and to the determination of physicochemical and colligative properties of substance mixtures.

A number of mathematical procedures on the topic are known, for example regularised discriminance analysis (RDA), principal component analysis (PCR), the partially least squares method (PLS) or multiple linear regression (MLR).

The chemometry method is not restricted to application to spectroscopic data, but spectroscopic data can be generated particularly easily and quickly if they are suitable for the characterisation of substance or system properties.

According to an advantageous embodiment of the inventive idea, it is provided that an optical spectral measurement is carried out on the measurement sample in the liquid state. The optical spectral measurement preferably includes a measurement of the fluorescence spectrum. Instead of a fluorescence spectrum, it is also possible to measure other optical properties of the measurement sample, such as, for example, emission spectra, absorption spectra, reflection spectra or. The sensitivity of the optical spectrometers used for this purpose can be in the optical wavelength range, but also, in addition or instead, cover the infrared range or the ultraviolet wavelength range. In addition, it is also conceivable to use spectroscopic measurements to record the X-ray range or high-energy radiation or even detectable corpuscular radiation.

According to another, likewise advantageous embodiment of the inventive idea, it is provided that a solid-phase extract with organically bound halogens be taken from the measurement sample, and an optical spectral measurement be carried out on the solid-phase extract. The optical spectral measurement can then advantageously include a measurement of the reflection spectrum or fluorescence spectrum.

According to yet another embodiment of the inventive idea, it is provided that a oscillatory device having a known inherent frequency be introduced into the measurement sample, where the oscillatory device consists of a material which preferably adsorbs organically bound halogens, and that, after a pre-specified time, the oscillatory device is removed and the change in the inherent frequency is determined. Suitable oscillatory devices can be, for example, electromagnetic oscillating circuits or alternatively vibrating masses, such as, for example, quartz resonators or the like.

The inherent frequency of a oscillatory device may be dependent, inter alia, on the geometry or mass coverage of a oscillatory structure. Its inherent frequency changes, in particular, if the surface or the inert mass is changed by coatings with substance. The special feature of the proposed method is based on the construction of the oscillatory device from a material which has a particular affinity for the substances to be determined (analytes), which are suitable as indicator for the pollutants to be determined.

There are some metals or conductive elements which are suitable as material for the oscillatory device since they have a specific, in some cases highly specific, affinity to prominent analytes, for example gold for mercury, silver or copper for halogens or transition metals for chalcogens.

The amount of adsorbed analyte causes a change in the inherent frequency of the oscillatory device which is proportional to this amount. By means of an external oscillating circuit of variable frequency, the inherent frequency of the oscillatory device can be determined before and after exposure thereof in the measurement sample to be investigated.

Since the resonance amplitude is proportional to the fourth power of the difference between the inherent vibration frequencies of the oscillatory device and the external oscillating circuit, the resonance amplitude supplies a signal which reacts very sensitively to changes in the analyte coverage on the oscillatory device and thus to the analyte concentrations in the measurement sample.

In order to facilitate determination of the concentration of adsorbable organically bound halogens, i.e. of an AOX sum parameter, it is provided that any requisite conversion of the determined concentration of organically bound halogens into a concentration of adsorbable organically bound halogens is carried out in a final method step.

The methods described above are faster and thus less expensive than the DIN methods known from the prior art. Compared with the prior art, no adsorption step and no combustion process are necessary. The methods can be fully automated

The flexibility of the evaluation and of the spectral range of the spectrometer usually employed means that these methods can be adapted to many analysis problems. Known examples are, inter alia, AOX, BTEX, alcohols, peroxides, metals, aldehydes, ketones, polymers, aromatics, functionalised aromatics or polyaromatics.

The invention also relates to an automatic measuring device. The known methods require a multiplicity of method steps, which, at acceptable costs, can hitherto only be performed manually and are therefore only carried out at time intervals of days or the like. Owing to the different instruments and devices, automation of the known methods would be associated with very considerable costs, which are in an unfavourable ratio to the savings arising in the operating costs incurred for the method.

An automatic measuring device according to the invention for the automatic measurement of organically bound halogens has a device for taking an amount of sample from a flow system, a device for conversion of the amount of sample into a measurement sample, a device for carrying out spectral measurements on the measurement sample and an evaluation device for evaluation of the result of the spectral measurements using reference measurements in order to determine the concentration of organic halogens.

In order to increase the accuracy, the measuring device can have a device for the enrichment of organically bound halogens in the measurement sample.

It is preferably provided that the device for enrichment is an extraction device.

In order to increase the detection sensitivity, it is provided that the measuring device has a device for the derivatisation of organically bound halogens in the measurement sample. Suitable derivatisation of the substances to be detected enables their optical activity to be stimulated or increased.

Likewise in order to increase the accuracy, it is provided that the measuring device has a device for the removal of inorganic halogens from the measurement sample.

It is advantageously provided that the evaluation device has a storage unit for reference measurements and an evaluation unit for comparison of the spectral measurement with reference measurements by means of chemometric methods in order to be able to determine the concentration of organically bound halides present in the waste water.

According to an embodiment of the inventive idea, it is provided that the device for carrying out a spectral measurement has an optical spectrometer.

The optical spectrometer has, for example, a device for the measurement of a fluorescence spectrum. It is also conceivable that the spectrometer has a device for the measurement of an X-ray fluorescence spectrum, a reflection spectrum, an emission spectrum or a transmission spectrum.

The optical spectrometer has preferably been adapted for carrying out measurements on liquid measurement samples.

It is advantageously provided that the measuring device has a device for conversion of the measurement sample into a solid-phase extract with organically bound halogens and that the optical spectrometer has a device for the measurement of a reflection spectrum. In this way, the concentration of, for example, organically bound halogens adsorbed onto active carbon can be determined rapidly and in an automatable manner and thus inexpensively without subsequent conversion being necessary.

By means of, for example, a commercially available adsorption apparatus for automatically increasing the concentration to be determined, a pre-specified volume of the measurement sample is passed through compacted active carbon or mixed with active carbon, stirred and filtered. The amount of substance adsorbed on the active carbon is freed from inorganic halide by washing. The adsorption enables the concentration of organically bound halogens to be increased by several orders of magnitude and thus the accuracy of subsequent measurements to be improved. For the spectral measurement, measurement of the reflection spectrum or fluorescence spectrum is then advantageously carried out.

According to another advantageous embodiment of the inventive idea, it is provided that the measuring device has a oscillatory device having a known inherent frequency which can be introduced into the measurement sample for a pre-specified duration, where the oscillatory device consists of a material which preferably adsorbs organically bound halogens, and that a change in the inherent frequency of the oscillatory device can be measured using the device for carrying out spectral measurements.

In order to facilitate conversion of the measured concentrations into comparable or officially stipulated concentrations, it is provided that the measuring device has a conversion unit by means of which at least one conversion of the determined concentration of organically bound halogens that may be necessary into a concentration of adsorbable organically bound halogens can be carried out.

In order to be able to automate the measurement methods described above as completely as possible, it is provided that the measuring device has a device for the automatic provision of an amount of sample from a laboratory or from a flow system. It is likewise provided that the measuring device has a device for the automatic transfer of the measurement sample into the device for carrying out the spectral measurement.

It is preferably provided that the measuring device has a signal transmitter device by means of which a transmittable signal can be generated. In this way, the treatment plant can be controlled and, in particular, the feed can be rapidly diverted into a separate tank depending on the measured concentration of pollutants.

The methods and devices described above are also suitable for carrying out a determination of the concentration of other spectroscopically active substances, in particular the concentrations of AOX contributors, BTEX contributors, alcohols, peroxides, metals, aldehydes, ketones, polymers, aromatics, functionalised aromatics or polyaromatics.

Instead of the concentration determination in a liquid or solid measurement sample, the concentration determination described can also be carried out in essentially unchanged form on a gaseous measurement sample.

Illustrative embodiments of the inventive idea are explained in greater detail below and are shown in the drawing, in which:

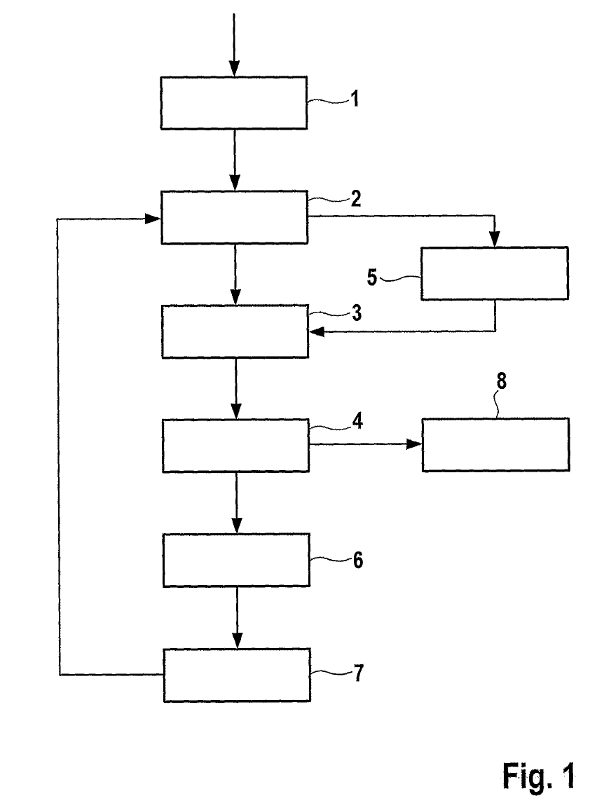

FIG. 1 shows a diagrammatic flow chart for the measurement of an AOX concentration in waste water and

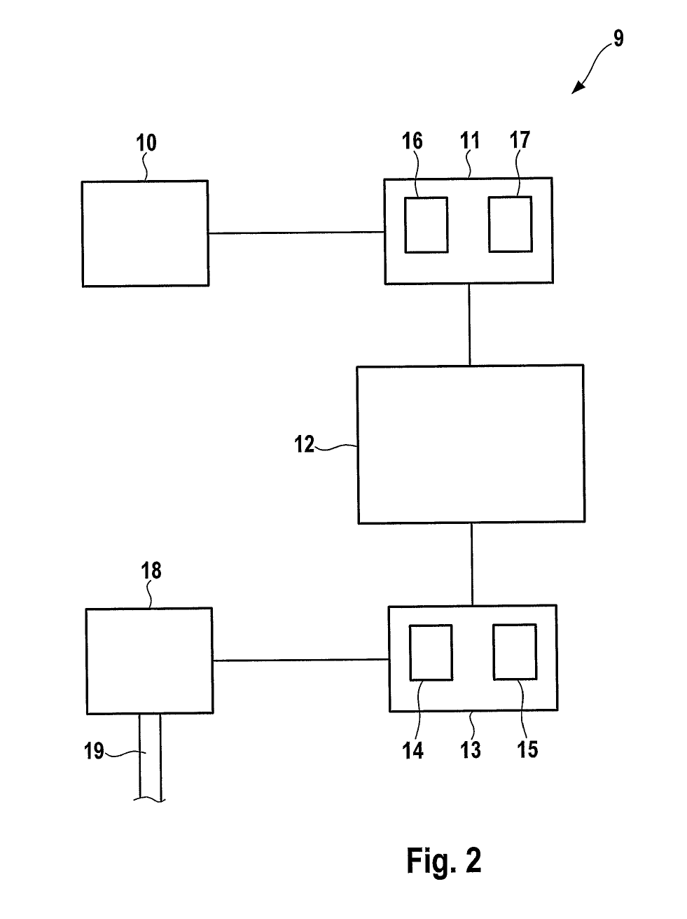

FIG. 2 shows a diagrammatic representation of a measuring device used for carrying out the method shown in FIG. 1.

As shown diagrammatically in FIG. 1, a chemometric calibration model for the AOX concentration in waste water is created starting from known or pre-specified AOX concentrations in a preparatory method step 1. Further calibration models, for example for the BTEX concentration in waste water or the concentrations in suitable extracts of AOX- or BTEX-containing waste water, can likewise be created.

For a measurement, a pre-specified amount of waste water is taken in a first method step 2 and converted into a measurement sample. A spectral measurement is carried out on the measurement sample in a further method step 3. The result of the spectral measurement is compared with reference measurements or with the calibration models in a method step 4 in order to determine the concentration of organically bound halogens.

In order to improve the detection sensitivity, the concentration of the pollutants or pollutant indicators to be measured can be enriched. To this end, the measurement sample having an unknown AOX or BTEX concentration is extracted in an additional method step 5 after the sampling and before the measurement. In order to facilitate informative comparisons with the reference measurements or calibration models, these reference measurements must have been extracted in the same manner. A spectrum of the extract is then measured in method step 3, which has already been described, and can be evaluated by comparison with the corresponding calibration models.

The method proposed here functions in such a way that an optical spectrum of an organic extract of the sample to be investigated is evaluated in accordance with a chemometric calibration model in method step 4. Depending on the type of AOX contributor to be expected from the respective AOX emitter, the ultraviolet, visual or infrared spectral region or a combination thereof should be used for this purpose. It is also conceivable to extend the spectral measurements to X-rays or high-energy radiation.

The prerequisite for the use of spectroscopy is an adequate spectroscopic representation of substance or mixture properties. Organohalogen compounds or substances whose totality contributes to the AOX, or aromatics corresponding to BTEX have a substantial optical activity in the ultraviolet/visual spectral region (UV/Vis) or in the fluorescence. Their usually considerable dilution in the waste water often makes direct measurement more difficult. In particular, dilution of the spectrally active and sought substance represents a weakening of the signal that can be evaluated and results in an unfavourable signal/noise ratio during the measurements.

For example, the detection limit of chlorobenzene in water is about 10-5. If chlorobenzene is the only AOX contributor with a concentration of a few ppm, the sensitivity of UV/Vis spectroscopy in transmission is at least 1 order of magnitude too low.

This necessitates the enrichment step 5 described above, by means of which the analyte concentration in the measurement volume can be increased by about two to three orders of magnitude corresponding to the volumes of water and extractant used having a ratio of typically 10+2 to 10+3, and this analyte concentration can thus be made measurable. The enrichment can be carried out easily and quickly using process machines which are commercially available today.

In order to permit a comparison or a statement regarding the concentration of adsorbable organically bound halogens, an at least approximately correct conversion from the measured concentration of extractable organically bound halogens (EOX) to the desired concentration of adsorbable organically bound halogens (AOX) can be carried out in a further method step 6. This conversion can likewise be supported on comparative measurements carried out in a preparatory manner.

In order to enable subsequent measurement and evaluation in a quasi-continuous measurement operation, the measuring device used is cleaned and in particular freed from organically bound halogens in a final method step 7. A subsequent measurement can be begun immediately with method step 2 which has already been described above.

In order to enable automated monitoring and control of a water treatment plant, it may be provided, in a method step 8 immediately following the chemometric analysis in method step 4, to generate control signals for the sewage-works operation and to transmit them to the control devices of the water treatment plant.

An automated measuring device 9, shown in FIG. 2 merely by way of example, for the automatic measurement of organically bound halogens has a withdrawal device 10 for a pre-specified amount of sample from a flow system with waste water. By means of a conversion device 11, the amount of sample is converted into a measurement sample which can be used further. The measurement sample is fed directly, by means of the conversion device 11, to a device for carrying out spectral measurements 12 on the measurement sample. In the illustrative embodiment described, this is an optical spectrometer 12. The optical spectrometer 12 has a device (not shown) for the measurement of a transmitted-light spectrum, for example a measurement cell.

The spectrometer 12 is connected to an evaluation device 13 for evaluation of the result of the spectral measurements using reference measurements. The evaluation device 13 has a storage unit 14 for reference measurements already carried out and an evaluation unit 15 for comparison of the spectral measurement with stored reference measurements by means of chemometric methods in order to be able to determine the concentration of organically bound halides present in the waste water.

In order to increase the measurement accuracy, the measuring device 9 has a device for the enrichment 16 of organically bound halogens in the measurement sample. The device for enrichment 16 is an extraction device, which is advantageously integrated into the conversion device 11. The solvent used for the extraction is a halogen-free solvent.

The conversion device may likewise have a device 17 for the reduction or elimination of inorganically bound halogens.

The instruments and apparatuses described as in each case individual devices 10 and 11 or 13 to 17 may each be designed as a unit of a single laboratory robot adapted in a suitable manner. The devices 10 to 12 and 16 and 17 which come into contact with the measurement sample have either in each case separate cleaning devices (not shown) or can be fed to a respectively associated or common cleaning device (likewise not shown) in order to be cleaned after the taking and measurement of a measurement sample and to be prepared for a fresh measurement.

The measuring device has a signal transmitter device 18, by means of which a transmittable signal can be generated. The signal transmitter device 18 is connected by means of signal lines 19 to control devices (not shown) of the water treatment plant (likewise not shown).

1. Method for determining the concentration of organically bound halogens present in waste water, where a pre-specified amount of waste water is taken and converted into a measurement sample, a spectral measurement is carried out on the measurement sample, and the result of the spectral measurement is compared with reference measurements in order to determine the concentration of organically bound halogens.

2. Method according to claim 1, characterised in that the organically bound halogens are enriched in the measurement sample before the spectral measurement is carried out.

3. Method according to claim 2, characterised in that the organically bound halogens are enriched by extraction.

4. Method according to claim 1, characterised in that the optical properties of the organically bound halogens are influenced by suitable derivatisation thereof.

5. Method according to claim 1, characterised in that inorganically bound halogens are removed from the measurement sample.

6. Method according to claim 1, characterised in that the comparison of the spectral measurement with reference measurements is carried out using chemometric methods.

7. Method according to claim 1, characterised in that an optical spectral measurement is carried out on the measurement sample in the liquid state and the optical spectral measurement includes a measurement of the transmitted-light spectrum.

8. Method according to claim 1, characterised in that a solid-phase extract with organically bound halogens is taken from the measurement sample, and an optical spectral measurement is carried out on the solid-phase extract.

9. Method according to claim 8, characterised in that the optical spectral measurement includes a measurement of the reflection spectrum or fluorescence spectrum.

10. Method according to claim 1, characterised in that a oscillatory device having a known inherent frequency is introduced into the measurement sample, where the oscillatory device consists of a material which preferably adsorbs organically bound halogens, and in that, after a pre-specified time, the oscillatory device is removed and the change in the inherent frequency is determined.

11. Method according to claim 1, characterised in that any requisite conversion of the determined concentration of organically bound halogens into a concentration of adsorbable organically bound halogens is carried out in a final method step 6.

12. Automatic measuring device 9 for the automatic measurement of organically bound halogens having a device 10 for taking an amount of sample from a flow system, having a device 11 for conversion of the amount of sample into a measurement sample, having a device 12 for carrying out spectral measurements on the measurement sample, and having an evaluation device 13 for evaluation of the result of the spectral measurements using reference measurements in order to determine the concentration of organic halogens.

13. Automatic measuring device 9 according to claim 12, characterised in that the measuring device 9 has a device 16 for the enrichment of organically bound halogens in the measurement sample.

14. Automatic measuring device 9 according to claim 13, characterised in that the device 16 for enrichment is an extraction device.

15. Automatic measuring device 9 according to claim 12, characterised in that the measuring device 9 has a device for the derivatisation of organically bound halogens in the measurement sample.

16. Automatic measuring device 9 according to claim 12, characterised in that the measuring device 9 has a device 17 for the removal of inorganically bound halogens from the measurement sample.

17. Automatic measuring device 9 according to claim 12, characterised in that the evaluation device 13 has a storage unit 14 for reference measurements and an evaluation unit 15 for comparison of the spectral measurement with reference measurements by means of chemometric methods in order to be able to determine the concentration of organically bound halides present in the waste water.

18. Automatic measuring device 9 according to claim 12, characterised in that the device 12 for carrying out a spectral measurement has an optical spectrometer.

19. Automatic measuring device 9 according to claim 18, characterised in that the optical spectrometer has a device for the measurement of a transmitted-light spectrum, a reflection spectrum or a fluorescence spectrum.

20. Automatic measuring device according to claim 12, characterised in that the measuring device 9 has a device for conversion of the measurement sample into a solid-phase extract with organically bound halogens.

21. Automatic measuring device 9 according to claim 12, characterised in that the measuring device 9 has a oscillatory device having a known inherent frequency which can be introduced into the measurement sample for a pre-specified duration, where the oscillatory device consists of a material which preferably adsorbs organically bound halogens, and in that a change in the inherent frequency of the oscillatory device can be measured using the device for carrying out spectral measurements.

22. Automatic measuring device 9 according to claim 12, characterised in that the measuring device has a conversion unit by means of which a conversion of the determined concentration of organically bound halogens that may be necessary into a concentration of adsorbable organically bound halogens can be carried out.

23. Automatic measuring device 9 according to claim 12, characterised in that the measuring device has a device for the automatic provision of an amount of sample from a laboratory or from a flow system.

24. Automatic measuring device 9 according to claim 12, characterised in that the measuring device 9 has a device 11 for the automatic transfer of the measurement sample into the device 12 for carrying out the spectral measurement.

25. Automatic measuring device 9 according to claim 12, characterised in that the measuring device 9 has a signal transmitter device 18 by means of which a transmittable signal can be generated.

26. Use of the device according to claim 12 for carrying out a determination of the concentration of other spectroscopically active substances, in particular the concentrations of AOX contributors, BTEX contributors, alcohols, peroxides, metals, aldehydes, ketones, polymers, aromatics, functionalised aromatics or polyaromatics.
